(19) United States
(12) Patent Application Publication
(10) Pub. No.: US 2004/0219192 A1
(43) Pub. Date: Nov. 4, 2004

(54) Devices and methods for heat-pulse assisted thermal applications of active subtances

(76) Inventors: Michael Horstmann, Neuwied (DE); Stefan Bracht, Ochtendung (DE)

Correspondence Address: BIRCH STEWART KOLASCH & BIRCH, PO BOX 747, FALLS CHURCH, VA, 22040-0747, US

(21) Appl. No.: 10/470,017

(22) PCT Filed: Jan. 14, 2002
(86) PCT No.: PCT/EP02/00286

(30) Foreign Application Priority Data
Jan. 23, 2001 (DE) 101 02 817.2

(51) Int. Cl. A61K 9/70

(52) U.S. Cl. 424/449

(57) ABSTRACT

The invention relates to devices for administering heat pulses to the skin, and for heat pulse-promoted transdermal administration of active substances, said device having one or more electric heating elements and a source of current as well as a control unit, the said control unit being suitable for causing a pulse-like heating of the said heating element.

The invention relates to devices for transdermal application of active substances, said devices causing a local heating of the application site in order to improve permeation. More particularly, the invention relates to devices of the afore-mentioned type containing an active substance-comprising transdermal therapeutic system (TTS), as well as to devices of the afore-mentioned type which are free of active substance, for administering heat pulses to human or animal skin.

The invention further comprises processes for transdermal administration of active substances wherein active substance permeation is increased by raising the temperature.

Transdermal therapeutic systems (TTS) are devices or administration forms which deliver one or more medicinal substances continuously, at a predetermined rate and over a predetermined period of time, to a pre-determined application site, i.e. to a particular area of skin. Compared to oral administration forms, TTS are advantageous because they allow administration of systemically active substances while avoiding the gastro-intestinal tract, whereby metabolising and inactivation, whose set otherwise occurs very quickly as a result of the “first-pass effect, is avoided or delayed. This results in an improved exploitation of active substance. Furthermore, it is possible in this way to avoid certain side effects which in oral administration can occur frequently. In addition, it is of advantage that these systems enable continuous and constant delivery of active substances, so that the blood plasma level is maintained at a largely constant level.

The treatment of various diseases with systemic medicaments which enter the body through the skin has meanwhile become widely known and described. A number of medicinal substances, nicotine, nitroglycerin, clonidine, estrogen and gestagens, for example, are already on the market in the form of transdermal therapeutic systems.

The structure of a TTS typically comprises the following components:

an active substance-containing reservoir;

a backing layer on the side averted from the skin, connected to said reservoir;

a pressure sensitive adhesive layer for attaching the system on the skin (this can be omitted if the active agent-containing reservoir is self-adhesive);

a detachable protective layer which covers the pressure-sensitive adhesive skin-contact side and which is detached prior to application.

The active substance-containing reservoir may be bag-shaped or constitute a solid matrix.

In the case of a bag-shaped reservoir, the back side of the flat bag is impermeable to the active substance, and the skin-contact side of the bag is formed by a permeable membrane. In such a system, the active substance is present in the form of a liquid or semi-solid preparation inside the bag.

In the case of TTS of the “matrix” type, the active substance is present, as a solution or dispersion, within a suitable polymer matrix serving as active substance reservoir. Materials used as pressure-sensitive base polymers for the matrix layer(s) are, for example, acrylate polymers or acrylate copolymers, polyisobutylenes, styrene-isoprene block copolymers or polysilicones. If necessary, the active substance-containing matrix may also be made up of a plurality of layers.

In the above described TTS, the active substance release to the skin takes place in principle by way of passive diffusion. A big problem with respect to the delivery of pharmaceutical active agents through the skin consists in the limited capacity of the human skin to accept such substances at a sufficient dosage rate. This is also the main reason why currently there are only a relatively small number of active substances suitable for transdermal therapy. On the other hand, ways are known of increasing the permeation of active substances through the skin. This can be achieved, for example, by using permeation-enhancing substances, or by increasing the thermodynamic activity of the active substance in the active substance reservoir. However, with many active substances, these measures are not sufficient, which makes further ways of increasing permeation necessary.

Using additives, in addition has the disadvantage of possibly leading to intolerance phenomena occurring on the skin. There is therefore a desire to increase the skin permeation further or to increase it several times over, without damaging the skin or unpleasant sensations occurring, as is frequently the case when permeation-enhancing substances are employed.

It is in principle known that the permeation rate of active substances through the skin is higher when the temperature is increased. There are, however, only few possibilities to make use of this effect in praxis since the normal skin temperature of around 32° C. can be increased only insignificantly without the occurrence of unpleasant sensations. As a consequence, little use has heretofore been made of this possibility.

The perception of pain usually begins when the skin temperature is increased above a value of approximately 45° C. This strongly limits the possibilities of exploiting the temperature effect to improve permeation. Thus, the increase in permeation through increased temperature, confirmed in several scientific publications (e.g. Watanabe Y, Hongo S, Matsumoto M: “Evaluation of excised loach skin for studies on transdermal permeation of drugs in vitro”; Yakugaku Zasshi, 1989 September, 109:9, 656-661) could up until now not be realised in the therapeutic praxis.

The use of artificially generated heat on the skin is also problematic because the usual heat application devices as a rule have a high consumption of current and are therefore not available in a portable form. Nevertheless, various electric devices have been described with which the skin can be heated or cooled for a certain period. As a rule, these devices are utilized on patients for physiotherapeutic purposes (cp. U.S. Pat. No. 5,746,702 and U.S. Pat. No. 5,097,828). It was therefore the object of the present invention to provide a device which enables the transdermal administration of active substances, and which enables an increase in active substance permeation which is not due to the presence of permeation-enhancing substances and which avoids a painful damage or irritation of the skin. It was a further object of the invention to indicate a process which enables the transdermal administration of active substances with increased active substance permeation, as described above.

This object is surprisingly achieved with a device according to claim 1 or claim 2, respectively with a process according to claim 14 or 15, the subclaims relating to further, especially useful embodiments of the invention.

According to claim 1, the inventive device for the heatpulse-promoted transdermal administration of active substances comprises a transdermal therapeutic system having on the skin-facing side an active substance-permeable electric heating element or several such elements. In addition, the inventive device comprises a control unit connected to the heating element, with the said control unit causing a pulse-like heating of the said heating element.

The transdermal therapeutic system (TTS), which is a component of the device according to claim 1, contains, apart from the heating element(s), an active substance reservoir, and a backing layer which covers the system on the outside. The active substance reservoir is connected with the heating element(s) and lies above the heating element. The heating element(s) is/are in direct contact with the skin during application.

The present invention furthermore comprises a device for administering heat pulses to the skin according to claim 2. This device comprises a pressure sensitive adhesive medicinal patch for attaching the device on the skin, one or more electric heating elements connected with said patch, as well as a control unit connected with the said heating element(s), and a current source. By means of the said control unit, it is possible to bring about a pulse-like heating of the said heating element. This device according to claim 2 does not contain active substance, respectively no active substance-containing layer; it can be designated as an active substance-free medicinal heat-pulse patch. Its use in the heat pulse-supported active substance administration is such that initially an active substance-containing preparation is applied to a skin area, and subsequently, possibly following a duration of action, the device according to claim 2 is applied to said skin area, whereby the heating element(s) are in contact with the skin surface.

The embodiment variants described in the following in principle relate to both the device according to claim 1 and to the device according to claim 2. It is, however, pointed out that in the case of the devices according to claim 1, the heating elements must have active substance-permeable properties; in the case of the devices according to claim 2 this is not necessary.

The heating element(s) of the devices according to the invention is/are coupled to a current source as energy-supplying device; by means of an interposed control unit it is achieved that in the heating element or heating elements heat is generated and released in pulses, i.e. a pulse-like heating of the heating elements and of the skin surface in contact with these heating elements is brought about.

The pulse-like delivery of heat to the skin has the advantage that if the device is appropriately positioned in proximity to the surface, a high influence of the heat on the outer horny layer (stratum corneum) is achieved, without lower skin layers, which are particularly heat-sensitive, becoming damaged. Because of the locally and very rapidly released amount of heat, a heat impulse is created which shortly heats the outer layer of the stratum corneum as well as the active substance-containing layer—the active substance-containing reservoir—of the TTS preferably to more than 50° C. After a few seconds the heat is conducted away to the lower skin layers where, due to the heat compensation, it creates only a slight sensation of heat, which is not perceived to be painful.

The heating of the heating element during the pulsed heating lies in the temperature range of between 40° C. and 200° C., preferably in the range between 50° C. and 120° C. The temperatures (target temperatures) indicated relate to the heating element. Due to the direct contact of the heating element to the skin, it is assumed that the above-mentioned temperatures can, for a short time, also be reached in the upper horny layer of the skin.

A preferred embodiment of the invention provides that to generate the heat pulses, a current intensity of 1 to 100 A is applied at an applied voltage of from 1.2 to 24 V over a pulse duration of 0.01 to 0.5 s at a time. Especially preferred are current intensities in the range of 5 to 20 A at voltages in the range of 1.2 to 7.2 V over a pulse duration of 0.05 to 0.1 s.

The duration of the heat pulses is limited and preferably amounts to 0.1 to 2 seconds, with particular preference 0.2 to 1 s. The temperature or/and the duration of the heat pulses is preset by the control unit or can be controlled by the same. When administering active substances using the device according to the invention, generally a plurality of successive heat pulses is released. Between the individual heat pulses there are time intervals during which the dissipation of the generated local heat into the lower skin layers can take place. The duration of these time intervals is at least 0.01 seconds and they can last up to several hours, e.g. up to 10 h. With preference, the time intervals between the individual pulses last 0.1 to 100 s, especially 1 to 60 s.

The individual parameters which characterize the heat pulses can be influenced by means of the above-mentioned control unit. These parameters can be fixedly preset at the beginning of the application; moreover, such embodiments are also provided wherein a subsequent or continuous control of or alternation of these parameters is possible. The said parameters are: temperature or temperature range (target temperature); duration of the individual heat pulses; length of the time intervals between the individual heat pulses; total number of heat pulses; maximum total duration of the treatment with heat pulses.

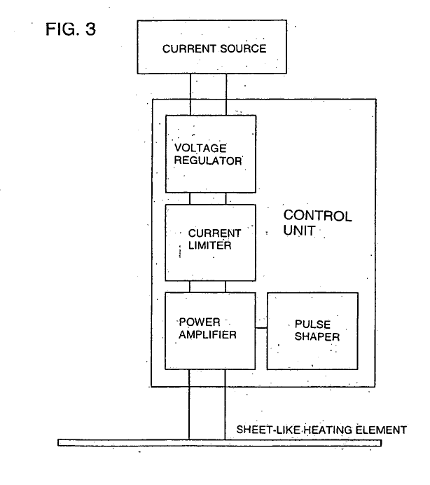

In addition to the above-mentioned properties of the control unit, it may also enable the control of the flow of current with respect to voltage, current intensity and/or the course in time. For example, it can also be achieved by means of the control unit that the shape of the pulse-like controlled flow of current corresponds to a rectangle, triangle, saw tooth or sine wave form. The schematic structure of such a control unit is described by way of example in FIG. 3.

The devices according to the invention can furthermore be equipped with miniaturized heat detectors which enable a control measurement of the temperature reached in the heating element or on the skin surface. For example, platinum temperature probes of SMD (“surface mounted device”) type and in thin layer technology which have a low heat capacity can be used as heat detectors.

The construction of control units in the form as described above is sufficiently known to the experts in electrotechnology. Particular store must be put on a power amplifier which, in the safe low voltage region of maximally ca. 24 V, allows the control of large flows of current of up to 100 Ampere and more at least for a short period of time. In this power stage, power transistors, field-effect transistors, or so-called IGBTs (insulated gate bipolar transistor) are used with preference, especially preferred are several of such components connected in parallel. An example for such power stages which are already integrated with pulse-forming control elements are so-called speed controllers, which are available, for example, for car, aircraft and ship model-making.

In a portable system, the source of current and the control unit are preferably, due to their size, spatially separated from the sheet-like heating element, which is in contact with the TTS. The source of current and the control unit may be worn as a separate unit at the belt, on one's wrist, or carried in pockets of one's clothing, for example. The cable connection between the control unit and the TTS should be constructed from a material with a specific resistance that is as low as possible, so that large currents can be led through small cable cross-sections. With preference, stranded wires of gilded silver, or pure gold wire are taken into consideration.

As a source of current for generating the heat pulses, preferably a galvanic battery or an accumulator, with particular preference a nickel-cadmium accumulator, or a capacitor are utilized. Apart from these, nickel-metal hydride (NiMH) accumulators and lithium ion accumulators as well as lithium batteries are suitable.

Because of the overall very low energy uptake of the inventive device, the current conduction device may also be equipped so as to be transportable. In a typical energy distribution, a source of current, for example, discharges with an energy content of 1000 Ws during operation, respectively the duration of application, 20 times, each time with an energy amount released of 50 Ws. In principle, any electric source of energy is suitable which has sufficient capacity. In the case of accumulators, the latter preferably amounts to 0.1 to 10 ampere-hours (Ah) at voltages of 1.2 to 24 V. For capacitors, it is preferably 0.01 to 10 F in the same voltage range.

As heating elements, those materials and structures are in principle suitable which are electrically conductive and possess a heat capacity which is as low as possible. Only in this way can it be ensured that the heat pulses are transmitted in small quanta, i.e. in the form of short and exact pulses, as immediately and unaltered as possible, to the skin surface. A heating element with a higher heat capacity would distort, that is, falsify the pulse shape of the heat generation.

In the case of the devices according to claim 1, it is furthermore required for the heating element(s) to be active substance permeable so that the active substance can reach the skin surface from the active substance reservoir.

The heating element of the device according to the invention is preferably sheet-like or layer-shaped, and with particular preference a thin, sheet-like, metal film; it may further be provided that the heating element is made up of several individual such film surfaces. The said metal film is preferably made of a metal selected from the group comprising copper, tin, aluminium and other soft metals, as well as alloys from the mentioned metals. Especially preferred is an embodiment of the metal film as utilized in the manufacture of metal film resistors having resistance values of from 0.1 to 10 ohm. As model examples of such heating elements may be considered resistors in SMD (Surface Mounted Devices) design, which are available as standard articles in electronics specialist shops (e.g. the firm of Conrad Elektronik, of Hirschau, Del.). Such components can be assembled in groups on a platinum surface.

In addition it is provided that the heating element(s) are made of an electrically conductive plastics film, or of a plurality of individual surface parts of such films. A polymer film high-enriched with carbon powder is preferably utilized for this purpose.

If the materials, e.g. metal films, of which the heating element is manufactured are active substance-impermeable, these materials may be provided with perforations, holes or slots, or made like a lattice, or subdivided into a plurality of strips lying side by side. In this way, the required permeability is created.

In a further embodiment of the invention it is provided that a flat-shaped winding of a thin wire, or a plurality of such windings, be used as the heating element. Preferably, wires of copper, tin, aluminium or other soft metals, as well as alloys of the afore-mentioned metals can be used for this purpose.

In the individual case it may also prove advantageous for the inventive devices to contain combinations of different heating elements which are made of different materials, as indicated above. Also, a single heating element may contain a combination of the afore-mentioned materials suitable for this purpose.

The area of the heating element of the inventive devices is, in the case of the devices of the type mentioned in claim 1, preferably, to the highest possible degree, identical with the area of the TTS connected therewith. With preference said area may amount to 5 to 100 cm², with particular preference 20 to 50 cm².

The heating element should as far as possible be in direct contact with the skin surface in order to transmit the shape and, in particular, the shortness of the heat pulse undamped to the surface of the skin. This can be accomplished by measures known to the skilled artisan; e.g. by an additional pressure-sensitive adhesive affixation layer, provided on the side of the heating element or TTS which is averted from the skin, or by a pressure-sensitive adhesive patch. Affixation of the inventive device on the skin may also be made possible, for example, by providing a backing layer of the TTS which projects beyond the surface of the active substance reservoir or of the sheet-like heating element, this margin area being provided, on the skin-facing side, with a pressure-sensitive adhesive coating.

The heating element's permeability to active substance is, if necessary, achieved preferably by providing pores, holes, slots or fine recessions in the heating element which allow active substances from the active substance-containing matrix to enter the heating element and thereafter the skin. The active substance can then, by way of cross-wise diffusion, accumulate in the uppermost skin layers and thereafter, during the heat pulse, overcome the barrier.

The proportion of free surface (i.e. pores, holes, slots or recessions) preferably amounts to 30-70%, relative to the overall surface area of the heating element. The maximal width of the closed surface portions is preferably in the range of 100-500 μm since such distances can be overcome by the active substance molecules by way of cross diffusion within a short period of time.

The inventive device according to the basic type described in claim 1 is in principle suited in connection with the most varied types of TTS constructions as have been described at the beginning hereof. In any case, there is located an active substance-permeable heating element or an active substance-permeable heatable layer on the side facing the skin, i.e. the release side of the TTS.

The materials suitable for the manufacture of the active substance reservoir, respectively of the active substance matrix, are basically known to the skilled artisan, likewise are the methods of manufacture. As base materials for the active substance matrix are suited, above all, pressure-sensitive polymers, e.g. acrylate polymers or acrylate copolymers, polyisobutylenes, styrene-isoprene block copolymers or polysilicones, or even suitable mixtures of polymers, or hot-melt adhesives.

The materials suitable for making the backing layer of the TTS and the detachable backing layer are likewise known to those skilled in the art. For example, sheets of polyvinyl chloride, ethylene vinyl acetate, vinyl acetate, polyethylene, polypropylene or cellulose derivatives may be used. For the detachable protective layer, basically the same materials may be used as for the backing layer, provided that said layer is subjected to an appropriate surface treatment; e.g. fluorosiliconization, so that it is detachable from the pressure-sensitive adhesive layer it covers and can be peeled off prior to application of the TTS. In addition, other materials may be used as detachable protective layers, too, such as, for example, polytetrafluoroethylene-treated paper, cellophane, polyvinyl chloride or similar materials.

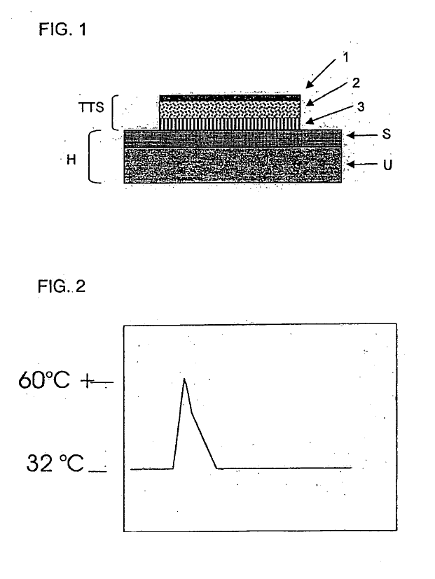

The invention will be described in more detail by means of the figures (FIG. 1 to 3).

FIG. 1 shows, by way of example, the structure of a TTS according to the present invention, and its arrangement on the skin (H) during application (in section), with

(1) indicating the backing layer

(2) indicating the active substance-containing matrix, and

(3) indicating the active substance-permeable layer of the TTS, which layer is heatable by heat pulses.

(S) designates the stratum corneum of the skin,

(U) the hypodermis.

FIG. 2 shows, in graphic representation, the time course of the temperature in the stratum corneum of the skin during application of a heat pulse-supported TTS according to the present invention. The temperature in the stratum corneum during application increases pulse-like from the normal skin temperature of 32° C. to the target temperature of 60° C.

FIG. 3 shows a schematic block diagram of the device according to the invention. The assembly comprises a source of current, connected with the control unit; the latter is in turn connected to the heating element of the device. The control unit is provided with a voltage regulator, a current limiter, a power amplifier, and a pulse shaper. The arrangement indicated is only by way of example and does not restrict the invention in any way.

The present invention also refers to processes for the transdermal administration of active substances which are based on the heating of the application site in pulses during the period of application in order to improve active substance permeation, whereby a temperature of at least 40° C. is achieved. Preferably, this is achieved by using a device according to the invention according to claim 1 or 2, as above-described.

The device for topical or transdermal administration of active substances to human or animal skin according to claim 14 provides for a device to be applied to the skin, which device comprises a transdermal therapeutic system. From the active substance reservoir of this TTS, the active substance(s) is/are released during the time of application, with the active substance permeation through the skin being improved by means of pulse-like temperature increases.

According to a further embodiment (claim 15), the process for topical or transdermal administration of active substances to human or animal skin may, however, also be performed in such a manner that

in a first step, the active substance(s) is/are applied to an area of skin in the form of a preparation of active substance, and

in a second step, a device is applied to the above-mentioned skin area by means of which the application site is heated in pulses during the period of application in order to improve active substance permeation.

According to this process, the administration of the active substance, that is, of the heat pulses is carried out sequentially, whereas in the process according to claim 14 the active substance and the heat pulses are administered simultaneously.

The above-mentioned active substance preparation can be present as a solution, ointment, cream or gel, for example. However, it can also be an active substance-containing TTS different from the active substance-free heat pulse patch described in step 2.

After application of the active substance or the active substance-containing preparation (step 1) and possibly after waiting for a certain incubation or action period (10 s to 24 h, preferably 1 min to 1 h) on the skin, the excess residues of the active substance preparation are superficially removed from the application site, and the active substance-free heat pulse patch is subsequently applied. In this manner, a homogenous and completely surface-congruent application of active substance and heat pulses can be achieved.

As heat pulse patch, preferably a device according to claim 2, optionally also-according to one of the variants described in the subclaims, may be utilized.

By means of the present invention, an increase of the skin permeation of transdermally administered active substances is made possible, independently of the use of permeation-enhancing additives. If a further increase in skin permeation is required, the heat pulse promoted administration may also be combined with the use of known skin permeation-enhancing substances.

Theoretically, with heat pulse-promoted administration, one, can expect an increase in skin permeation by a factor of 2 to 100, preferably from 5 to 20.

The invention may in principle be utilized for the topical or transdermal administration of the most varied active substances. The term “active substances” is understood to mean any medicinal substances used in human or veterinary medicine, including vitamins, enzymes and hormones, as well as active substances for cosmetic treatments.

The use of the inventive device is of particular advantage for the administration of poorly skin-permeable substances such as, for example, peptides, proteins, nucleotides, polynucleotides, or for immunising suitable biomolecules or sections of such biomolecules. Furthermore, especially such active substances are to be taken into consideration which, due to their molecular mass of more than 500 Da or due to their melting points above 200° C., are little suited for the use in passive TTS.

The device according to the present invention and the process according to the present invention are advantageously suitable for the administration of medicaments for the purpose of therapeutic treatment of humans or animals.

1. Device for heat pulse-supported transdermal administration of active substances, comprising
a transdermal therapeutic system (TTS), having a backing layer, an active substance-containing reservoir and, on the side facing the skin, an active substance-permeable electric heating element or a plurality of such elements,
and a control unit, connected to said heating element, and a current source, said control unit being suitable for causing pulse-like heating of said heating element.

2. Device for administration of heat pulses to human or animal skin, comprising
a pressure-sensitive adhesive medicinal patch;
one or more electric heating element(s) connected with the patch;
and a control unit, connected with said heating element(s), and a current source, said control unit being suitable for effecting pulse-like heating of the said heating element.

3. Device according to claim 1, characterized in that the heating of the heating element/heating elements takes place in the temperature range of between 40° C. to 200° C., preferably in the range of between 50° C. and 120° C., and that the duration of the heat pulses is 0.1 to 2 seconds, preferably 0.2 to 1 s, the temperature and/or the duration of the heat pulses being preset by the control unit, or being controllable.

4. Device according to claim 1, characterized in that it produces the said heat pulses repeatedly, the length of the time intervals between the individual heat pulses being at least 0.01 s, preferably 0.1 s to 100 s, with particular preference 1 to 60 s, and the duration of these time intervals being preset or controllable by the control unit.

5. Device according to claim 1, characterized in that as a source of current, one or more galvanic battery/batteries or one or more accumulator(s), preferably selected from the group of the nickel-cadmium accumulators, nickel-metal hydride (NiMH) accumulators, lithium ion accumulators and lithium batteries, or one or more capacitor(s), is/are used, the electric capacitance in the case of accumulators preferably being 0.1 to 10 Ah at voltages of from 1.2 to 24 V, and in the case of capacitors in the same voltage range, preferably 0.01 to 10 F.

6. Device according to claim 1, characterized in that the heating element is sheet-like or layer-shaped and preferably is a thin, sheet-like metal film or made up of a plurality of such individual film surfaces, the said metal film preferably being made of a metal selected from the group comprising copper, tin, aluminium: and other soft metals as well as alloys of the said metals.

7. Device according to claim 6, characterized in that the heating element is made up of individual, thin, sheet-like metal films, with metal film resistors preferably being used as the metal films.

8. Device according to claim 6, characterized in that the metal film is provided with perforations, holes or slots, or that it is configured like a lattice, or is divided into a plurality of strips lying next to one another.

9. Device according to claim 1, characterized in that as the heating element a flat-shaped winding of a thin wire, or a plurality of such windings, is used, the said wire preferably being made of a metal selected from the group comprising copper, tin, aluminium and other soft metals, as well as alloys of the said metals.

10. Device according to claim 1, characterized in that the heating element consists substantially of an electrically conductive plastics film, or of a plurality of individual surface parts of such films, with a polymer film high-enriched with carbon powder being used with preference.

11. Device according to claim 1, characterized in that the control unit is provided with a speed controller, a BEC control circuit board, as well as a pulse generator.

12. Device according to claim 1, characterized in that during a pulse length of 0.01 to 0.5 s, preferably from 0.05 to 0.1 s, it generates current intensities in the range of from 1 to 100 A, preferably from 5 to 20 A, at an applied voltage of 1.2 to 24 V, preferably from 1.2 to 7.2 V.

13. Use of a device according to claim 1 for topical or transdermal administration of medicaments or other active substances for the purpose of therapeutic or cosmetic treatment of humans or animals.

14. Process for topical or transdermal administration of active substances to human or animal skin wherein one or more active substances are released from an active substance reservoir of a transdermal therapeutic system, and are delivered to the skin, characterized in that, during the period of application, the application site is heated in a pulsed manner to improve active substance permeation, with a temperature of at least 40° C. being reached.

15. Process for topical or transdermal administration of active substances to human or animal skin, characterized in that
in a first step, the active substance(s) is/are applied to an area of skin in the form of an active substance preparation, and
in a second step, a device is applied to the above-mentioned skin area by means of which the application site is heated in a pulsed manner during the period of application in order to improve active substance permeation, with a temperature of at least 40° C. being reached.

16. Process according to claim 15, characterized in that the second step is performed at a distance in time of 10 s to 24 h, preferably 1 min to 1 h, after the first step.

17. Process according to claim 14, characterized in that the pulse-like heating of the application site is brought about by an electric heating element, or a plurality of such elements, which is/are in contact with the skin during the period of application, the said heating element being connected with a source of current and a control unit.

18. Process according to claim 14, characterized in that the pulse-like heating takes place in the temperature range of between 40° C. and 200° C., preferably in the range between 50° C. and 120° C., and that the duration of the heat pulses is 0.1 to 2 seconds, preferably 0.2 to 1 s.

19. Process according to claim 14, characterized in that the pulse-like heating is performed repeatedly, with the length of the time intervals between the individual heat pulses being at least 0.01 s, preferably 0.1 to 100 s, with particular preference 1 to 60 s.

20. Process according to claim 14, characterized in that the temperature or temperature range reached during the pulse-like heating, and/or the duration of the individual heat pulses, and/or the length of the time intervals between the individual heat pulses, and/or the total number of heat pulses, and/or the maximum total duration of the treatment with heat pulses, are each preset or subsequently altered or adjusted by a control device connected with the heating element or heating elements and the source of current.

21. Process according to claim 14, characterized in that the heat pulses are generated by current intensities in the range of from 1 to 100 A, preferably from 5 to 20 A, at an applied voltage of from 1.2 to 24 V, preferably 1.2 to 7.2 V, during a pulse length of from 0.01 to 0.5 s, preferably from 0.05 to 0.1 s.

22. (cancelled)
